(19) United States
(12) Patent Application Publication
(10) Pub. No.: US 2012/0010433 A1
(43) Pub. Date: Jan. 12, 2012

(54) DEVICE AND PROCESS FOR SOLID/LIQUID SEPARATION OF SOLID-LIQUID SUSPENSIONS

(75) Inventors: Christian Münnich, Leverkusen (DE); Karl-Heinz Köhler, Aachen-Brand (DE); Stefan Westernacher, Kempen (DE)

(73) Assignee: Bayer MaterialScience AG, Leverkusen (DE)

(21) Appl. No.: 13/178,009

(22) Filed: Jul. 7, 2011

(30) Foreign Application Priority Data
Jul. 9, 2010 (EP) EP10169032.9

(51) Int. Cl. B01D 39/16; C07C 37/70; B01D 33/06

(52) U.S. Cl. 568/720; 210/359; 210/402; 210/767; 210/505

(57) ABSTRACT

In a device and a process for continuous solid/liquid separation (filtration) of solid-liquid suspensions on moving filters, for example on rotating drum filters or belt filters, the active filtering layer contains a woven fabric material composed of synthetic fibres, which has an enhanced filtrate permeability and also an enhanced thermal stability with improved dimensional stability, and also improved mechanical strength compared with polypropylene fibres.

### PRIORITY

Priority is claimed to European Patent Application No. 10169032.9, filed Jul. 9, 2010. The disclosure of the aforementioned priority document is incorporated herein by reference in its entirety.

### BACKGROUND

The field of the present invention relates to devices and processes for continuous solid/liquid separation (filtration) of solid-liquid suspensions on moving filters, for example on rotating drum filters or belt filters, wherein the active filtering layer contains a woven fabric material composed of synthetic fibres, which has an enhanced filtrate permeability and also an enhanced thermal stability up to 130° C. with improved dimensional stability, and also improved mechanical strength compared with polypropylene fibres. The woven fabric material contains fibres selected from the group of ethylene-chlorotrifluoroethylene copolymers (E-CTFE), polytetrafluoroethylene (PTFE) and polyetheretherketone (PEEK), and is secured on the outside surface of the filter drum using clamping devices operating according to the groove-tongue principle. The service life of the woven filter fabric, until wear and tear makes it necessary to replace this woven filter fabric, is distinctly enhanced over a conventional woven filter fabric of polypropylene (PP).

The filtration of suspensions of bisphenol A (BPA)-phenol adduct crystals in liquid phenol on vacuum drum filters is known and is described in WO 2001/046105 A1 for example. The thermal stability of the filter medium is referred to therein, among other aspects, without suitable filtering materials being specified, however.

Thermally stable filtering materials composed of PTFE, or modified PTFE, and of copolymers of ethylene with chlorotrifluoroethylene (E-CTFE), and also the production of filters from such materials are already known and are described in U.S. Pat. No. 5,213,882 A for example. However, this reference is concerned with fibrous nonwoven webs, the properties of which are not necessarily comparable to woven fabrics formed from such fibre materials. Nor does the reference mention mechanical stabilities or permeabilities of such filters.

Filtering materials formed from PEEK fibres are also known and described in WO 99/19043 A1 for example. However, this reference does not reveal whether woven filtering materials are concerned and which mechanical properties or permeabilities such filters have.

There is accordingly a need for filter cloths which, for the same or a reduced breakthrough of solids, provides an enhanced throughput of filtrate and at the same time, compared with known filter cloths in woven polypropylene, for constantly recurring cleaning purposes, have an enhanced thermal stability to steam of 2-5 bar pressure, and also sufficient dimensional stability at these temperatures and improved mechanical strengths. In the case of rotating drum filters, this means more particularly that, during the operating life of the filter cloth, no cracks appear at the places securing the filter cloth on the filter drum, which are configured according to the groove-tongue principle and which, owing to the higher angular deflections in the region of the securement, can represent a particular mechanical load on or a weakpoint of the filter cloth. The mechanical strength of the filter cloth is important because drum filters are continuously in movement and subjected to different pressures, and because, furthermore, the resulting filter cake is continuously being scraped off the cloth surface mechanically. This mechanical stress is, in contradistinction to static filters, of appreciable import for the useful life of the filter cloth; it should be extended by about 1 year, compared with the polypropylene filter cloths hitherto used, to about 5 years. Similarly, the filtrate permeability of the improved filter cloth should be at least equivalent to that of polypropylene filter cloths, or even be better than that perhaps.

### SUMMARY OF THE INVENTION

The problem addressed by the devices and processes described herein is accordingly that of providing an improved filter cloth for moving filters for example rotating drum filters, or belt filters, for continuous filtration of suspensions of bisphenol-phenol adduct crystals in liquid phenol having the abovementioned advantages over the prior art filter cloths. More particularly, the filter cloths described herein serve to distinctly enhance the permeability to the phenol filtrate coupled with equivalent or even improved solids breakthrough.

The inventive problem was solved, surprisingly, by using specifically woven filter cloths with warp threads and weft threads of PEEK or fluorine-containing ethylene copolymers having similar or even reduced air permeabilities compared with the polypropylene filter cloths used hitherto. Surprisingly, these filter cloths when used in the filtration of suspensions of bisphenol-phenol adduct crystals in liquid phenol on moving filters have a higher permeability to the phenol filtrate than the polypropylene filter cloths used hitherto, even though the air permeability of the filter cloths, when measured in [L/dm²×min], is less than that of the polypropylene filter cloths. In addition to the improved thermal stability, the filter cloths have better mechanical stability and distortion resistance at elevated temperature.

Bisphenol herein is a bis(4-hydroxyaryl)alkane.

The device and process described herein provide for continuous filtration of solid-liquid suspensions on continuous filtration apparatuses, more particularly with moving filters and preferably on rotating drum filters, more particularly for continuous filtration of suspensions of bisphenol-phenol adduct crystals in liquid phenol on moving filters. The device consists of a conventional moving filter and the filter cloth on this moving filter and more particularly described hereinbelow. The process consists in using the above device for continuous filtration of suspensions of bisphenol-phenol adduct crystals in liquid phenol which are generated in an operation to manufacture bisphenol.

### DETAILED DESCRIPTION OF THE PREFERRED EMBODIMENTS

TSF vacuum drum filter from Krauss Maffei, described in WO 2001/046105 A1, is an example of moving filters that may be used. Preferably, such a drum filter contains as filter cells a cake-forming zone, a washing zone, a dry suction zone, an aeration zone and optionally a cake removal zone and a cloth rinsing zone. The filter cloth then lies on the filter drum on the filtrate-permeable supporting element for the filter cloth, and is tensioned and secured there with ropes composed of synthetic fibres or preferably corrosion-resistant metallic springs or a combination thereof by the tongue-groove principle. The end faces of the drum filter are preferably not utilized as filtering areas. The functioning of such a vacuum drum filter is elucidated at length in WO 2001/046105 A1. However, other filtering arrangements are usable, for example pressure rotation filters, belt filters, disc filters, plate filters or flat filters.

The filter cloths, for a vacuum drum filter for example, are woven fabrics from the group consisting of ethylene-chlorotrifluoroethylene copolymers (E-CTFE), polytetrafluoroethylene (PTFE) and polyetheretherketone (PEEK), preferably woven fabrics made of E-CTFE. These filter cloths also have improved properties over metallic filtering cloths or filtering materials made of resistant metallic engineering materials.

The filter cloths are woven fabrics with warp threads and weft threads which may be woven for example in the twill weave or the plain reverse dutch weave or the satin weave; twill weave is preferred. The thickness of the fibres can vary from 300 to 1000 μm; preference is given to fibres from about 600 μm to 800 μm for a woven fabric weight of 500 to 600 g/m². The air permeability of the woven filter cloth fabric under a pressure of 20 mm water column can vary from 90 to 1500 [L/dm²×min], and air permeabilities of 500 to 1300 [L/dm²×min] are preferred. The filter cloth may optionally be calendered one or more times.

These filter cloths have high dimensional stability at elevated thermal loading at 130° C. over a period of 48 hours. Comparable filter cloths of polypropylene exhibit distinct shrinkages in the transverse and longitudinal directions of the filter belt under identical conditions. The filter cloths can therefore be cleaned with steam at higher temperature more intensively and hence at longer intervals, which enhances the time for which the entire filtering equipment is available.

Similarly, the water permeability of the filter cloths, as an additional measure of the permeability of the filter cloths, is less than for the comparable polypropylene filter cloths, and thus correlates with the air permeability. Despite the lower permeability, the filter cloths allow a higher throughput of the phenolic bisphenol A-containing suspensions on the same vacuum drum filter at otherwise identical operational parameters when the filter cloths are used instead of the comparative polypropylene filter cloths.

The filtration properties, as measured in terms of the bisphenol content of the filter cake, correspond to those of polypropylene filter cloths when the filter cloths used are used under comparable conditions.

The present process for continuous filtration of suspensions of bisphenol-phenol adduct crystals in liquid phenol consists in using moving filters by using the above-described filter cloths for separating these suspensions. This process, when compared with the prior art processes, such as the process described in WO 2001/046105 A1 for example, has the advantage of enhanced permeability, longer time availability due to reduced cleaning and inspection intervals for the filter cloth on the moving filter, due to avoidance of cracks and other mechanical damage to the filter cloth. This process has the further advantage of enhanced productivity, owing to the enhanced product throughput as a consequence of the improved filtration properties.

This process is part of a production process for bisphenol, described at length in WO 2001/046105 A1.

The bis(4-hydroxyaryl)alkanes are prepared continuously or batchwise, preferably continuously over a catalyst, preferably an ion exchanger in fixed bed reactors for condensation reactions.

Produced bis(4-hydroxyaryl)alkanes are for example those of the general formula (I),

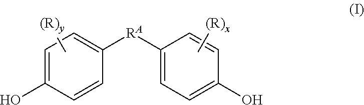

where
R^{A} represents a linear or branched C₁-C₁₈-alkylene radical, preferably C₁-C₆-alkylene radical, or a C₅-C₁₈-cycloalkylene radical, preferably a C₅-C₁₂-cycloalkylene radical,
R independently represent a linear or branched C₁-C₁₈-alkyl radical, preferably C₁-C₆-alkyl radical, a C₅-C₁₈-cycloalkyl radical, preferably a C₅-C₁₂-cycloalkyl radical, a C₆-C₂₄-aryl radical, preferably a C₆-C₁₂-aryl radical, or a halogen radical, and
x and y independently represent 0 or an integer from 1 to 4, preferably independently 0, 1 or 2.

Preferred bis(4-hydroxyaryl)alkanes are 2,2-bis(4-hydroxyphenyl)propane (bisphenol A (BPA)), 2,4-bis(4-hydroxyphenyl)-2-methylbutane, 2,2-bis(3-methyl-4-hydroxyphenyl)propane, bis(3,5-dimethyl-4-hydroxyphenyl)methane, 2,2-bis(3,5-dimethyl-4-hydroxyphenyl)propane, 2,4-bis(3,5-dimethyl-4-hydroxyphenyl)-2-methylbutane, 1,1-bis(4-hydroxyphenyl)cyclohexane and 1,1-bis(4-hydroxyphenyl)-3,3,5-trimethylcyclohexane (bisphenol TMC).

Particularly preferred bis(4-hydroxyaryl)alkanes are 2,2-bis(4-hydroxyphenyl)propane (bisphenol A (BPA)), 2,2-bis(3,5-dimethyl-4-hydroxyphenyl)propane, 1,1-bis(4-hydroxyphenyecyclohexane and 1,1-bis(4-hydroxyphenyl)-3,3,5-trimethylcyclohexane (bisphenol TMC).

Very particular preference is given to 2,2-bis(4-hydroxyphenyl)propane (bisphenol A).

Bis(4-hydroxyaryl)alkanes are obtainable in a conventional manner by reacting aromatic monohydroxy compounds that are not substituted in the p-position with ketones that have at least one aliphatic group on the carbonyl function, in a condensation reaction. The intermediate product preferably obtained is an adduct of bis(4-hydroxyaryl)alkane and the aromatic monohydroxy compound used as starting material and subsequently separated into the desired bis(4-hydroxyaryl)alkane and aromatic monohydroxy compound.

Suitable aromatic monohydroxy compounds are for example those of the general formula (II),

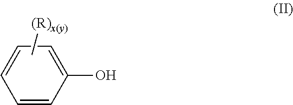

which are not substituted in the p-position and in which
R independently represent a linear or branched C₁-C₁₈-alkyl radical, preferably C₁-C₆-alkyl radical, a C₅-C₁₈-cycloalkyl radical, preferably a C₅-C₁₂-cycloalkyl radical, a C₆-C₂₄-aryl radical, preferably a C₆-C₁₂-aryl radical, or a halogen radical and
X or y represent 0 or an integer from 1 to 4, preferably 0, 1 or 2.

Examples of suitable aromatic monohydroxy compounds of the general formula (II) are for example phenol, o- and m-cresol, 2,6-dimethylphenol, o-tert-butylphenol, 2-methyl-6-tert-butylphenol, o-cyclohexylphenol, o-phenylphenol, o-isopropylphenol, 2-methyl-6-cyclopentylphenol, o- and m-chlorophenol or 2,3,6-trimethylphenol. Preference is given to phenol, o- and m-cresol, 2,6-dimethylphenol, o-tert-butylphenol and o-phenylphenol, and very particular preference is given to phenol.

Suitable ketones are for example those of the general formula (III),

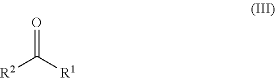

where
R¹ represents a linear or branched C₁-C₁₈-alkyl radical, preferably C₁-C₆-alkyl radical, and
R² represents a linear or branched C₁-C₁₈-alkyl radical, preferably C₁-C₆-alkyl radical, or a C₆-C₂₄-aryl radical, preferably a C₆-C₁₂-aryl radical, or
R¹ and R² together represent a linear or branched C₂-C₁₈-alkyl radical, preferably C₂-C₁₂-alkyl radical.

Examples of suitable ketones of the general formula (III) are acetone, methyl ethyl ketone, methyl propyl ketone, methyl isopropyl ketone, diethyl ketone, acetophenone, cyclohexanone, cyclo-pentanone, methyl-, dimethyl- and trimethylcyclohexanones which may each also have geminal methyl groups, e.g. 3,3-dimethyl-5-methylcyclohexanone (hydroisophorone). Preferred ketones are acetone, acetophenone, cyclohexanone and its methyl-bearing homologues, particular preference being given to acetone.

C₁-C₆-Alkyl represents for example methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl or 1-ethyl-2-methylpropyl, C1-C18-alkyl further represents for example n-heptyl and n-octyl, pinacolyl, adamantyl, the isomeric menthyls, n-nonyl, n-decyl, n-dodecyl, n-tridecyl, n-tetradecyl, n-hexadecyl, n-octadecyl or stearyl.

C₁-C₆-Alkylene/C₁-C₁₈-alkylene represents for example the alkylene groups corresponding to the preceding alkyl groups.

C₅-C₁₂-Cycloalkyl represents for example cylopentyl, cyclohexyl, cyclooctyl or cyclododecyl.

Examples of C₆-C₂₄-aryl or C₆-C₁₂-aryl are phenyl, o-, p-, m-tolyl, naphthyl, phenanthrenyl, anthracenyl or fluorenyl.

Halogen can represent fluorine, chlorine, bromine or iodine, preferably fluorine or chlorine and more preferably chlorine.

The examples which follow serve for exemplary elucidation of the invention and are not intended to limit the scope of the claims. Examples identified as “comparative” examples do not represent embodiments of the invention. All other examples are presented to reflect the inventive concepts described herein.

### EXAMPLES

The inventive examples utilized the following filter cloths:

### SEFAR TETEXMONO 08-1033-W 115 (E-CTFE)

### SEFAR TETEXMONO 17-2032-W 155 (PEEK)

where E-CTFE denotes ethylene-chlorotrifluoroethylene copolymers and PEEK denotes polyetheretherketone.

The filter cloth of the type PP2763 (1200 L/(dm²·min)), consisting of polypropylene (PP), from Verseidag was used for the comparative examples, as well as the metal filter of the type SPW40; mesh 80×400 from 1.4306 (X2CrNi19-11, AISI 304L) from Haver & Boecker.

Water permeability was determined in a laboratory apparatus specially built for this purpose. A defined quantity (5000 ml) of temperature-controlled water was initially charged into a temperature-controlled hydrostatic column above a vertical measuring sector through which flow is to take place. The filter cloth to be tested was installed in the vertical measuring sector through which flow is to take place in a defined free cross-sectional area such that the main flow resistance of the measuring assembly is predetermined by the filter cloth installed in the defined cross-sectional area through which flow is to take place. The quantity measured in filter cloth testing is the time needed by the water quantity to flow out of the temperature-controlled hydrostatic column.

### Examples 1 to 6, presented in detail in what follows, are summarized in the Table 1 which follows.

TABLE 1
Comparative overview of examples

| Example | 1 | 2* | 3 |
|---|---|---|---|
| filtration | rotary filter | rotary filter | suction filter |
| manufacturer | Sefar | Verseidag | Sefar |
| filter cloth type | TETEXMONO | PP2763 | TETEXMONO |
| | 08-1033-W 115 | | 08-1033-W 115 |
| filter material | E-CTFE | PP | E-CTFE |
| air permeability in L/(dm² min) | 1080 | 1200 | 1080 |
| water permeability/run-through | 121 s | 102 s | 121 s |
| time | | | |
| dimensional stability in air | yes | shrinkage by | yes |
| at 130° C. for 48 h | | about 10% in x | |
| | | and y dimensions | |
| rotary filter: | 8.8% by weight | 9.0% by weight | N.A. |
| bisphenol A content of filtrate | | | |
| (continuous filtration) | | | |
| suction filter: | N.A. | N.A. | 9.4% |
| bisphenol A content of filtrate | | | |
| (manual filtration on suction filter) | | | |
| Example | 4 | 5* | 6* |
| filtration | suction filter | suction filter | suction filter |
| manufacturer | Sefar | Verseidag | Haver & Boecker |
| filter cloth type | TETEXMONO | PP2763 | Filtertyp SPW40; |
| | 17-2032-W 155 | | mesh 80 × 400 |
| filter material | PEEK | PP | 1.4306 |
| air permeability in L/(dm² min) | 1200 | 1200 | |
| water permeability/run-through | 124 s | 102 s | 129 s |
| time | | | |
| dimensional stability in air at | yes | shrinkage by | yes |
| 130° C. for 48 h | | about 10% in x | |
| | | and y dimensions | |
| rotary filter: | N.A. | N.A. | N.A. |
| bisphenol A content of filtrate | | | |
| (continuous filtration) | | | |
| suction filter: | 9.4% | 9.6% | 10.0% |
| bisphenol A content of filtrate | | | |
| (manual filtration on suction filter) | | | |

*comprarative

### Example 1

The BPA/phenol adduct crystals generated in the acid-catalyzed reaction of phenol and acetone with subsequent suspension crystallization are separated from the liquid phase via a rotary filter and forwarded for further purification. To this end, a solids content in the feed stream of 25% by weight and a feed temperature into the rotary filter of 41° C. are set. Filtration takes place on a phenol-resistant, thermally stable filter cloth TETEXMONO 08-1033-W 115 from Sefar having an air permeability of 1080 L/dm²/min. The vacuums are 100 mbar in the cake-forming zone, 300 mbar in the washing zone and 300 mbar in the dry suction zone. The rotary filter housing is inertized with nitrogen under a slight overpressure of 10 mbar. Drum rotary speed, filter cake thickness, circuit nitrogen rate and the aspirating openings in the control disc are set such that the residual moisture content of the filter cake is <15% by weight based on the mixed crystal quantity. The rotary filter operates stably at a feed stream of up to about 3.3 t/(h·per m² of filter area).

The rinsing of the filter cake in the washing zone utilizes pure phenol having a temperature of 55° C., the rinse quantity for the filter cake cleaning being 100%, based on the filter cake quantity.

The filter cloth rinse utilizes phenol having a temperature of 80° C., the rinse quantity for cloth rinsing being 80% by weight, based on the amount of filter cake. This manner of filtration provides a BPA-phenol adduct having high purities (>99% without phenol fraction).

The bisphenol A content of the filtrate is about 8.8% by weight.

### Example 2 (Comparative)

Example 1 is repeated except that the same rotary filter is equipped with PP2763 filter cloth from Verseidag, in contrast to Example 1.

Under otherwise identical operating parameters, the rotary filter can be operated stably up to a maximum feed stream of up to about 2.7 t/(h·per m² of filter area). A higher feed stream leads to a continuous increase in the suspension level in the rotary filter trough, and would eventually lead to complete flooding of the apparatus.

This manner of filtration likewise provides a BPA-phenol adduct having high purities (>99% without phenol fraction).

The bisphenol A content of the filtrate is about 9.0% by weight.

### Example 3

The BPA-phenol adduct crystals generated in the acid-catalyzed reaction of phenol and acetone with subsequent suspension crystallization are separated from the liquid phase via a temperature-controlled suction filter. The filtration, which was carried out batchwise, was used to set a solids content of 25% by weight in the suspension and a suspension temperature of 41° C. The suction filter is likewise temperature controlled to 41° C. The filtration was carried out on a phenol-resistant, thermally stable TETEXMONO 08-1033-W 115 filter cloth from Sefar having an air permeability of 1080 L/dm²/min. About 2 m³ of BPA-phenol adduct crystal-containing suspensions are used per m² of filter cloth area. The vacuum in the cake-forming zone is about 100 mbar.

The filter cake is washed with pure phenol temperature controlled to 60° C., the rinse quantity for the filter cloth cleaning being 100%, based on the filter cloth quantity.

The washed filter cake is sucked dry down to a residual moisture content of about 15% and analyzed.

This manner of filtration provides a BPA-phenol adduct having high purities (>99% without phenol fraction).

The bisphenol A content of the filtrate is about 9.4% by weight.

### Example 4

The BPA-phenol adduct crystals generated in the acid-catalyzed reaction of phenol and acetone with subsequent suspension crystallization are separated from the liquid phase via a temperature-controlled suction filter. The filtration, which was carried out batchwise, was used to set a solids content of 25% by weight in the suspension and a suspension temperature of 41° C. The suction filter is likewise temperature controlled to 41° C. The filtration was carried out on a phenol-resistant, thermally stable TETEXMONO 17-2032-W 155 filter cloth from Sefar having an air permeability of 1200 L/dm²/min. About 2 m³ of BPA-phenol adduct crystal-containing suspensions are used per m² of filter cloth area.

The vacuum in the cake-forming zone is about 100 mbar. The filter cake is washed with pure phenol temperature controlled to 60° C., the rinse quantity for the filter cloth cleaning being 100%, based on the filter cloth quantity. The washed filter cake is sucked dry down to a residual moisture content of about 15% and analyzed.

This manner of filtration provides a BPA-phenol adduct having high purities (>99% without phenol fraction).

The bisphenol A content of the filtrate is about 9.4% by weight.

### Example 5

The BPA-phenol adduct crystals generated in the acid-catalyzed reaction of phenol and acetone with subsequent suspension crystallization are separated from the liquid phase via a temperature-controlled suction filter. The filtration, which was carried out batchwise, was used to set a solids content of 25% by weight in the suspension and a suspension temperature of 41° C. The suction filter is likewise temperature controlled to 41° C. The filtration was carried out on a phenol-resistant, thermally stable PP2763 filter cloth from Verseidag having an air permeability of 1200 L/dm²/min. About 2 m³ of BPA-phenol adduct crystal-containing suspensions are used per m² of filter cloth area.

The vacuum in the cake-forming zone is about 100 mbar. The filter cake is washed with pure phenol temperature controlled to 60° C., the rinse quantity for the filter cloth cleaning being 100%, based on the filter cloth quantity. The washed filter cake is sucked dry down to a residual moisture content of about 15% and analyzed.

This manner of filtration provides a BPA-phenol adduct having high purities (>99% without phenol fraction).

The bisphenol A content of the filtrate is about 9.6% by weight

### Example 6 (Comparative)

The BPA-phenol adduct crystals generated in the acid-catalyzed reaction of phenol and acetone with subsequent suspension crystallization are separated from the liquid phase via a temperature-controlled suction filter. The filtration, which was carried out batchwise, was used to set a solids content of 25% by weight in the suspension and a suspension temperature of 41° C. The suction filter is likewise temperature controlled to 41° C. The filtration was carried out on a phenol-resistant, thermally stable SPW40 metal filter, mesh 80×400, construction material 1.4306 from Haver & Boecker. About 2 m³ of BPA-phenol adduct crystal-containing suspensions are used per m² of filter cloth area.

The vacuum in the cake-forming zone is about 100 mbar. The filter cake is washed with pure phenol temperature controlled to 60° C., the rinse quantity for the filter cloth cleaning being 100%, based on the filter cloth quantity. The washed filter cake is sucked dry down to a residual moisture content of about 15% and analyzed.

This manner of filtration provides a BPA-phenol adduct having high purities (>99% without phenol fraction).

The bisphenol A content of the filtrate is about 10.0% by weight.

1-10. (canceled)

11. A device for continuous filtration of solid-liquid suspensions on continuous filtration apparatuses, wherein the active filtering layer contains a woven material with warp threads and weft threads formed from synthetic fibres selected from the group consisting of ethylene-chlorotrifluoroethylene copolymers (E-CTFE), polytetrafluoroethylene (PTFE) and polyetheretherketone (PEEK).

12. The device according to claim 11, wherein the active filtering layer lies on a cylindrical side of a moving filter.

13. The device according to claim 12, wherein the filtration takes place on rotating drum filters.

14. The device according to claim 11, wherein a thickness of the fibres is in the range from 300 to 1000 μm.

15. The device according to claim 11, wherein air permeability of the woven material is in the range from 90 to 1500 [L/dm²×min] at a pressure of 20 mm water column.

16. A process for continuous filtration of solid-liquid suspensions on continuous filtration apparatuses which comprises utilizing a filtering layer containing a woven material with warp threads and weft threads of synthetic fibres selected from the group consisting of ethylene-chlorotrifluoroethylene copolymers (E-CTFE), polytetrafluoroethylene (PTFE) and polyetheretherketone (PEEK).

17. The process according to claim 16, for continuous filtration of suspensions of bisphenol-phenol adduct crystals in liquid phenol.

18. A process for isolating bisphenol-phenol adduct crystals from a suspension of bisphenol-phenol adduct crystals in liquid phenol by filtration of this suspension with the device according to claim 11.

19. The process according to claim 18, wherein the bisphenol conforms to the following formula (I): 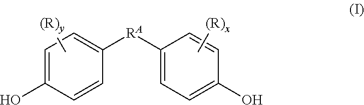
where
R^{A} represents a linear or branched C₁-C₁₈-alkylene radical or a C₅-C₁₈-cycloalkylene radical,
R independently represent a linear or branched C₁-C₁₈-alkyl radical, a C₅-C₁₈-cycloalkyl radical, a C₆-C₂₄-aryl radical or a halogen radical, and
x and y independently represent 0 or an integer from 1 to 4.

20. The process according to claim 19, wherein the bisphenol is 2,2-bis(4-hydroxyphenyl)propane (bisphenol A (BPA)), 2,2-bis(3,5-dimethyl-4-hydroxyphenyl)propane, 1,1-bis(4-hydroxyphenyl)cyclohexane or 1,1-bis(4-hydroxyphenyl)-3,3,5-trimethylcyclohexane (bisphenol TMC).
